(19) United States
(12) Patent Application Publication
(10) Pub. No.: US 2025/0134820 A1
(43) Pub. Date: May 1, 2025

(54) PHARMACEUTICAL COMPOSITIONS

(71) Applicant: Novartis AG, Basel (CH)

(72) Inventors: Saket BHASIN, Hyderabad (IN); Swati KULKARNI, Hyderabad (IN)

(21) Appl. No.: 18/701,740

(22) PCT Filed: Oct. 19, 2022
(86) PCT No.: PCT/IB2022/060061

§ 371 Date: Apr. 16, 2024

(30) Foreign Application Priority Data
Oct. 21, 2021 (IN) 202111047878

(51) Int. Cl. A61K 9/48; A61K 9/16; A61K 31/4747

(52) U.S. Cl. CPC A61K 9/4866; A61K 9/1623; A61K 9/1635; A61K 9/1694; A61K 9/4858; A61K 31/4747

(57) ABSTRACT

Pharmaceutical compositions for oral administration comprising the drug substance (S)-1′-chloro-8-(difluoromethoxy)-8′,8′-difluoro-6-(trifluoromethyl)-7′,8′-dihydro-3H,6′H-spiro[imidazo[1,2-]pyridine-2,5′-isoquinoline], or a pharmaceutically acceptable salt thereof, or a free form thereof are described. Further, processes for preparing said pharmaceutical 5 compositions for oral administration and uses of said pharmaceutical compositions in the manufacture of a medicament are described.

### FIELD OF THE INVENTION

The present invention relates to the field of pharmacy, particularly to pharmaceutical compositions for oral administration comprising the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof.

The present invention also relates to a process for preparing said pharmaceutical composition for oral administration; and to the use of said pharmaceutical composition in the manufacture of a medicament.

### BACKGROUND OF THE INVENTION

(S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof, is a compound having the structure of formula

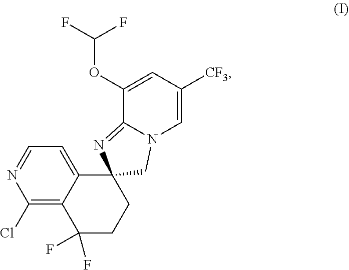 [SMILES: FC(F)OC1=CC(C(F)(F)F)=CN2C[C@@]3(CCC(F)(F)c4c3ccnc4Cl)N=C12]

or Compound A, which is disclosed, in PCT/IB2021/053486 under Example 31, and is incorporated by reference in its entirety. Compound A is an inhibitor of hypoxia-inducible factor-2α (HIF2α) and is useful in the treatment of conditions, disease and disorders mediated by HIF2α (e.g., cancerous conditions and disorders).

Several crystalline free forms and salts forms of the compound and methods for preparing said forms were also described in PCT/IB2021/053486 and are incorporated by reference in its entirety herein.

There is a need to formulate Compound A into pharmaceutical compositions, especially oral pharmaceutical formulations, such that the therapeutic benefits of the compound may be delivered to a patient in need thereof. Posing a challenge resolving this need is the physiochemical properties of the therapeutic compound. An object of the present invention is to provide an exemplary solution by making a pharmaceutical composition in the form of a solid oral dosage form that may be ingested by a patient.

### BRIEF DESCRIPTION OF THE FIGURES

FIG. 1 shows the dissolution profile for capsules comprising 150 mg of Compound A carried out with USP-I/basket/100 RPM/900 mL 0.01N HCl dissolution media volume and using USP-II/paddle/50 RPM/900 mL 0.01 N HCl dissolution media volume with sinker.

FIG. 2 shows the dissolution profile capsules comprising 12.5 mg and 25 mg of Compound A carried out with USP-I/basket/100 RPM/900 mL 0.01N HCl dissolution media volume.

FIG. 3 shows the dissolution profile for Hard Gelatin (HGC) and Hypromellose (HPMC) capsules comprising 12.5 mg of Compound A over stability in open petri-plate carried out in 900 mL of 0.1N HCl using paddles with sinker.

FIG. 4 shows the dissolution profile for capsules of Hard Gelatin (HGC) and Hypromellose (HPMC) comprising 150 mg of Compound A over stability in open petri-plate carried out in 900 mL of 0.1 N HCl using paddles with sinker.

FIG. 5 shows the process diagram for manufacturing 12.5 mg and 25 mg non-gelatin capsules of Compound A.

FIG. 6 shows the process diagram for manufacturing 100 mg non-gelatin capsules of Compound A.

### SUMMARY OF THE INVENTION

As every active pharmaceutical ingredient (API) has its own physical, chemical and pharmacological characteristics, a suitable pharmaceutical composition and dosage form has to be individually designed for every new API.

The drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof (herein referred as Compound A) is a highly potent active pharmaceutical ingredient (API). Compound A, in the free form, was found to have a developability classification system (DCS) of IIa (dissolution rate limited absorption) for doses up to 50 mg and DCS IIb (solubility rate limited absorption) fordoses greaterthan 50 mg. Compound A in the fumarate salt form was found to have a DCS IIa for doses up to 100 mg and DCS IIb for doses greater than 100 mg. The compound shows a favorable ADME/PK profile with low to very low clearance, good oral bioavailability leading to high and sustained exposure across tested species, and a low Q-Plus risk. However, the free form of Compound A was found to display low clearance and moderate to high oral bioavailability across tested species but has a poor solubility, a slow dissolution rate in biorelevant media, and the potential for a large proton pump inhibitor/acid-reducing agent (PPI/ARA) and positive food effect. GastroPlus modeling predicted dissolution and/or solubility limitations of the free form of Compound A at high doses. In comparison, the fumarate salt form of Compound A was found to have superior biopharmaceutical properties, especially in order to enable fast dissolution and maximum absorption and exposure up to high doses.

The design of a pharmaceutical composition, a pharmaceutical dosage form as well as a robust and economical pharmaceutical manufacturing process for the fumarate salt form of Compound A is especially difficult for (inter alia) the following reasons:

Being a salt, risk of disproportionation to the base is a critical parameter to be monitored during development and stability.

The pHmax of the fumarate salt is estimated to be about 4.5. An increase in pH above 4.5 may potentially increase the risk of disproportionation.

Consequently, excipients making up the pharmaceutical composition or any aqueous media used in the manufacture of the drug product that may increase the pH might cause chemical degradation of Compound A.

It is therefore difficult to design a pharmaceutical composition or a dosage form for Compound A that is stable and is of an acceptable size to be easily swallowable. It is moreover difficult to design a manufacturing process that provides an ease of scale up, a robust processing and economic advantages.

In view of the above-mentioned difficulties and considerations, it was surprising to find a way to prepare a stable pharmaceutical composition for oral administration comprising the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof (herein referred as Compound A). The pharmaceutical compositions are in the form of solid oral dosage forms, especially capsules. The capsules are filled with granules of the therapeutic compound blended with an inner phase comprising at least one pharmaceutically acceptable excipient.

Aspects, advantageous features and preferred embodiments of the present invention summarized in the following items, respectively alone or in combination, contribute to solving the object of the invention.

In accordance with a first aspect of the present invention, there is provided a capsule for oral administration comprising
(a) (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof,
(b) one or more fillers, and
(c) one or more disintegrants.

In accordance with a second aspect of the present invention, there is provided a pharmaceutical blend comprising
(a) (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof,
(b) one or more fillers, and
(c) one or more disintegrants,
   wherein said blend is manufactured by a dry process. Preferably, said dry process is a direct blending or a roller compaction process, more preferably, a roller compaction process.

In accordance with a third aspect, there is provided a dry process for making the capsules as defined by the first aspect comprising a roller compaction process step.

In accordance with a fourth aspect, there is provided a capsule obtainable by a roller compaction process according to the third aspect.

In accordance with a fifth aspect, there is provided a dry process for making the pharmaceutical blend as defined by the second aspect and for making a capsule by machine-encapsulation of said pharmaceutical blend comprising a roller compaction process step.

In accordance with a sixth aspect, there is provided a pharmaceutical blend obtainable by the dry process according to the fifth aspect and a capsule obtainable by said dry process further comprises an additional encapsulation step.

The above mentioned aspects provide the following advantages:

By the densification of the voluminous drug substance and the excipients by roller compaction (1) the blend in an amount corresponding to a dose up to 150 mg of Compound A can be filled into a capsule of size 0; and (2) it becomes feasible to fill the blend into capsules by machine; and (3) the drug becomes more easily swallowable by patients.

By the avoidance of a wet process (e.g., wet granulation), the potential for disproportionation of the drug substance is minimized.

### DETAILED DESCRIPTION OF THE INVENTION

Herein after, the present invention is described in further detail and is exemplified.

In the aspects of the present invention the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], herein also referred to as Compound A, is present in its free form or in the form of any pharmaceutically acceptable salt, complex, co-crystal, hydrate or solvate thereof.

In one embodiment, Compound A is present in its free base form. In another embodiment, Compound A is present as fumarate salt; in yet another embodiment as cinchonidine salt; in yet another embodiment as trifluoroacetate salt; in yet another embodiment as hydrochloride salt.

In one embodiment, Compound A is present as fumarate salt in a polymorphic form characterized by an XRPD (X-ray powder diffraction) pattern comprising a characteristic peaks (2 theta) at about of 24.9±0.2°, 6.2±0.2° and 20.9±0.2°; further comprising one or more characteristic peaks (2 theta) selected from peaks at about 10.9±0.2° and 18.5±0.2°; even further comprising one or more characteristic peaks (2 theta) selected from peaks at about 22.8±0.2°, 12.9±0.2° and 16.1±0.2° as described in PCT/IB2021/053486 as “Form A” of the fumarate salt. The latter disclosure provides the process for preparing this form and further details on the characterization of this form in Example 120 and is incorporated herein as reference.

In one embodiment, Compound A is present as free form in a polymorphic form characterized by an XRPD (X-ray powder diffraction) pattern comprising a characteristic peaks (2 theta) at about of 9.7±0.2°, 18.4±0.2° and 19.4±0.2°, further comprising one or more characteristic peaks (2 theta) selected from peaks at about 13.4±0.2° and 20.7±0.2°; even further comprising one or more characteristic peaks (2 theta) selected from peaks at about 24.2±0.2°, 22.1±0.2° and 10.3±0.2° as described in PCT/IB2021/053486 as “Form A” of the free form. The latter disclosure provides the process for preparing this form and further details on the characterization of this form in Example 120 and is incorporated herein as reference.

In the aspects of the present invention the drug substance, i.e. the Compound, is present in the pharmaceutical blend or in the content of the capsule in an amount of at least 3%, preferably 3-80%, 3-70%, 3-60%, 3-50%, or 3-40%, preferably 3.0-40%, 3.5-40%, or 3.8-40%, preferably 6 to 70%, 8 to 70%, 10 to 70%, 15 to 70%, 20 to 70%, preferably 6 to 62%, 8 to 62%, 10 to 62%, 15 to 62%, 20 to 62%, preferably 6.4±2%, 15.9±2%, 61.1±2% by weight of the drug substance in its free base form based on the total weight of the blend or of the content of the capsule, respectively. The amount values above refer to the drug substance as a fumarate salt.

In the aspects of the present invention, fillers (or diluents) include at least one of microcrystalline cellulose, calcium phosphate dibasic, cellulose, lactose, sucrose, mannitol, sorbitol, starch, and calcium carbonate. For example, the one or more fillers can be lactose and microcrystalline cellulose, more preferably cellulose MK GR.

The term “filler” or “diluent” is used herein in its established meaning in the field of pharmaceutics, e.g. provide bulk, for example, in order to make the pharmaceutical composition a practical size for processing, or aid processing, for example, by providing improved physical properties such as flow, compressibility, and hardness.

In the aspects of the present invention, the filler(s) is (are) present in the pharmaceutical blend or in the content of the capsule in an amount of 0.1-85%, 0.5-80%, 0.5-60%, 0.5-50%, 0.5-40%, 0.5-30%, or 0.5-20%, preferably 20-85% or 35-70%, more preferably 36% or 77% by weight based on the total weight of the blend or content of the capsule, respectively. The above mentioned ranges apply for all the fillers as listed above. Preferably, the filler is lactose and is present in an amount of 3-60% or 20-55%, preferably in an amount of 20±1%, 28±1% or 52±1%. Preferably, the filler is also Cellulose MK GR and is present in an amount of 9-30% or 12-25%, preferably in an amount of 15.7±1% or 25±1%.

In the aspects of the present invention, disintegrants include starch and its derivatives (e.g. low substituted carboxymethyl starches such as Primogel® by Generichem Corp., Explotab® by Edward Mendell Co., or Tablo® by Blanver), pregelatinized starches, potato, maize, and corn starches), clays (e.g. Veegum HV and bentonite), crosslinked cellulose and its derivatives (e.g. cross-linked form of sodium carboxymethylcellulose (CMC), e.g. as known under the brand names AcDiSol® by FMC Corp., Nymcel ZSX by Nyma, Primellose® by Avebe, Solutab® by Blanver), cross-linked polyvinylpyrrolidone (PVP XL) or polyvinyl polypyrrolidone (PVPP), e.g. as known under the brand names Crospovidone® by BASF Corp., Kollidon CL® by BASF Corp., Polyplasdone XL® by ISP Chemicals LLC. Preferably, the disintegrant is a cross-linked polyvinylpyrrolidone (polyvinyl pyrrolidone XL, crosslinked PVP or PVP XL) or polyvinyl polypyrrolidone (PVPP).

The term “disintegrant” is used herein in its established meaning in the field of pharmaceutics, e.g. as a facilitator to break up granules or tablets into smaller fragments when getting in contact with liquids to promote rapid drug dissolution.

In the aspects of the present invention the disintegrant(s) is (are) present in the pharmaceutical blend or in the content of the capsule in an amount of 0.5-50%, 1-30%, 1-25%, 1-20%, 1-15%, or 1-12%, preferably 1-12%, more preferably 1-8% by weight based on the total weight of the blend or content of the capsule, respectively. The above mentioned ranges apply for all the disintegrants as listed above. Preferably, the disintegrant is crosslinked PVP (PVP XL) and is present in an amount of 1-8%, 1-6%, 1-5%, 1-2%, preferably of 1.5±1%, 4.4±1% or 6±1%, even more preferably about 5% or about 6%.

All those percentage values are weight by weight percentage values and based on the total weight of the blend or content of the capsule.

According to the first aspect, the invention provides a capsule for oral administration comprising
(a) the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof,
(b) one or more fillers, and
(c) one or more disintegrants.

Said capsule may further comprise
(d) one or more lubricants, preferably magnesium stearate in an amount of 0.1-2%, preferably 1-1.5% by weight based on the total weight of the content of the capsule, and/or
(e) one or more glidants, preferably colloidal silicon dioxide (colloidal silica), more preferably AEROSIL® 200 PH, preferably in an amount of 0.1-1.5%, preferably 0.5-1% by weight based on the total weight of the content of the capsule.

The capsule may be a hard capsule or a soft capsule, preferably cellulose (HPMC) based or made out of gelatin and optionally comprising colorants, process aids (e.g. sodium lauryl sulfate), and/or preservatives. Preferably, the capsule is a hard non-gelatin HPMC capsule.

The inventors observed an increased an increase in degradation products when the composition contains HPMC capsule powder. This indicates that a desiccant made be necessary in the packaging of HPMC capsule compositions of Compound A.

The size of the capsule may range from 0 (body volume 0.69 mL), 1, 2, 3 or 4 (body volume 0.20 mL). Preferably, for the present invention a capsule of size 0 is used for a dosage strength of 150 mg, a capsule of size 1 is used for a dosage strength of 25 mg, a capsule of size 2 or 3 is used for a dosage strength of 12.5 mg. The sizes of the capsule herein refers to as the standardized sizes for two-pieces hard capsules in the pharmaceutical industry practice, e.g. capsule size “1” has a volume of about 0.5 mL, e.g. 0.48-0.50 mL, a locked length of about 19-20 mm e.g. 19.4 mm, and an external diameter of about 7 mm, e.g. 6.6 or 6.9 mm.

It is one of the advantages of the present invention, that a relatively small capsule sizes can be used, which is based on the densified pharmaceutical blend as described in further detail below, which allows to deliver the required high doses (e.g. up to 150 mg per unit) of the drug substance via easily swallowable dosage forms.

According to the second aspect, the invention provides a pharmaceutical blend comprising
(a) (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof,
(b) one or more fillers, and
(c) one or more disintegrants,
   wherein said blend is manufactured by a dry process. Preferably, said dry process is a direct blending or a roller compaction process, more preferably, a roller compaction process.

Due to the use of roller compaction, the voluminous drug substance can be densified to such an extent that at least 150 mg of the pharmaceutical blend of the present invention can be filled into a capsule of size 0 with a body volume of 0.69 mL or a capsule of smaller size.

Therefore, the bulk density of the pharmaceutical blend of the present invention is the “poured bulk density” before capsule filling and is at least 0.4 g/mL, 0.5 g/mL, 0.6 g/mL, 0.7 g/mL, 0.8 g/mL 0.9 g/mL, 1.0 g/mL, 1.1 g/mL or 1.2 g/mL. Alternatively, the bulk density of the pharmaceutical blend of the present invention is the “tapped bulk density” and is at least 0.5 g/mL, 0.6 g/mL, 0.7 g/mL, 0.8 g/mL 0.9 g/mL, 1.0 g/mL, 1.1 g/mL or 1.2 g/mL, preferably at least 0.7 g/mL, at least 0.8 g/mL, or at least 0.9 g/mL.

The “tapped bulk density” is often also referred to as “consolidated bulk density”, measured according to the standard methods as defined in Pharmacopeia, e.g. the European Pharmacopeia, using standardized equipment (e.g. 250 ml graduated cylinder (readable to 2 ml) with a mass of 220±44 g; and a settling apparatus capable of producing, in 1 minute, either nominally 250±15 taps from a height of 3±0.2 mm, or nominally 300±15 taps from a height of 14±2 mm. The support for the graduated cylinder, with its holder, has a mass of 450±10 g. According to said standard methods 500 and 1250 taps on the same powder sample (100 g) is carried out and the corresponding volumes V500 and V1250 are determined. If the difference between V500 and V1250 is less than or equal to 2 mL, V1250 is the tapped volume. If the difference between V500 and V1250 exceeds 2 ml, one has to repeat in increments such as 1250 taps, until the difference between succeeding measurements is less than or equal to 2 ml. The tapped bulk density is then the 100 g sample weight divided by the (final) V1250 volume.

As the inventors have surprisingly found that the application of a wet process (e.g., during mixing, compaction, milling, blending steps) results in disproportionation of the drug substance, it is important for the present invention to design a manufacturing process which avoids moisture during any mixing, compaction, milling, blending and/or compaction process step.

Accordingly, in the third aspect the present invention provides a dry process for making the capsules as defined by the first aspect of the invention comprising a dry compaction process step, preferably roller compaction.

More specifically, the dry process according to the third aspect is characterized by the following process steps:
(1) roller compaction of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof, with one or more fillers, and one or more disintegrants, and optionally one or more additional pharmaceutical excipients, to obtain granules,
(2) blending the granules of step 1 with additional pharmaceutical excipients, e.g. glidants (preferably colloidal silicon dioxide or AEROSIL® 200 PH) and lubricants (preferably magnesium stearate) and optionally further fillers or disintegrants (preferably PVP XL) to obtain a pharmaceutical blend,
(3) machine-encapsulation of the pharmaceutical blend of step 2 into capsules, preferably hard non-gelatin HPMC capsules.

In a fourth aspect, the capsules resulting from said process are provided.

The term “machine-encapsulation” is used herein to contrast the process of the present invention from any process in which the capsules are filled by hand or with the help of simple pieces of equipment (e.g. plastic plates with predrilled holes) and simple loading devices. With such bench-scale fillings only small quantities of capsules can be produced, typically up from 50 to 5,000 capsule per hour. Instead, “machine-encapsulation” herein refers to industrial-scale filling by machines like the auger filling machine using a ring system or the Zanasi as dosing tube or dosator-type machine or the Höfliger & Karg as dosing disc and tamping finger machine. With such semi-automatic to full-automatic machines capsules can be produced with outputs of typically 5000-150,000 capsules per hour (caps/h).

In accordance with a fifth aspect, there is provided a dry process for making the pharmaceutical blend as defined by the second aspect and for making a capsule by machine-encapsulation of said pharmaceutical blend comprising a roller compaction process step.

More specifically, the dry process according to the fifth aspect is characterized by the following process steps:
(1) roller compaction of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof, with one or more fillers, and one or more disintegrants, and optionally one or more additional pharmaceutical excipients, to obtain granules,
(2) blending the granules of step 1 with additional pharmaceutical excipients to obtain a pharmaceutical blend,

In addition, it is provided a dry process for making a capsule comprising the steps 1 and 2 according to the fifth aspect as described above and further comprising the step of
(3) machine-encapsulation of the pharmaceutical blend of step 2 into capsules, preferably hard non-gelatin HPMC capsules.

As a sixth aspect, there is provided a pharmaceutical blend obtainable by the dry process according to fifth aspect.

As a modification of the sixth aspect, there is provided a capsule obtainable by the dry process according to the fifth aspect including the machine-encapsulation step 3.

As a further aspect, there is provided a dose unit comprising the capsule of the first aspect or the pharmaceutical blend according to the second aspect in the form of a capsule. More specifically, the dose unit according to this further aspect comprises the drug substance, i.e. the Compound in its free base form in an amount of 1-150 mg, preferably 12.5-150 mg, more preferably 12.5 mg, 25 mg, 100 mg, or 150 mg.

As a further aspect, there is provided a capsule according to the first aspect wherein the size of the capsule is 0 and comprises up to 100 mg, or up to 125, or up to 150 mg, preferably up to 100 mg, more preferably 100 mg to 150 mg of drug, even more preferably 100 mg of the Compound or any of its pharmaceutical acceptable salt, wherein the drug dose is calculated in its free base form of the Compound.

As a further aspect, there is provided a capsule according to the first aspect wherein the capsule size is 1 and comprises up to 50 mg, more preferably 25 mg of the Compound or any of its pharmaceutical acceptable salt, wherein the drug dose is calculated in its free base form of the Compound.

As a further aspect, there is provided a capsule according to the first aspect wherein the capsule size is 2 and comprises up to 25 mg, preferably up to 12.5 mg of the Compound or any of its pharmaceutical acceptable salt, wherein the drug dose is calculated in its free base form of the Compound.

The following are preferred embodiments of the present invention: A capsule for oral administration comprising
(a) 3-62% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], present as fumarate salt,
(b) 20-85% by weight of lactose and cellulose, and
(c) 1-8% by weight crosslinked polyvinylpyrrolidone.
   based on the total weight of the content of the capsule.

A capsule for oral administration comprising
(a) 3-62% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], present as fumarate salt,
(b) 20-85% by weight of lactose and cellulose,
(c) 1-8% by weight crosslinked polyvinylpyrrolidone,
(d) 0.1-2% by weight magnesium stearate, and
(e) 0.1-1% by weight colloidal silicon dioxide,
   based on the total weight of the content of the capsule.

In a preferred embodiment, the range of the drug substance is 15.8-66.1%.

In a preferred embodiment, the range of lactose is 20.8-52.7%.

In a preferred embodiment, the range of cellulose is 15.6-25%.

In a preferred embodiment, the range of crosslinked polyvinylpyrrolidone is 5-6.1%.

In a preferred embodiment, the range of magnesium stearate is 1-1.5%.

In a preferred embodiment, the range of colloidal silicon dioxide is 0.5-0.85%.

In a very preferred embodiment, the present invention provides:

A capsule for oral administration comprising an inner phase and an external phase, the inner phase comprising:
(a) 3-62% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], present as fumarate salt,
(b) 20-85% by weight of lactose and cellulose,
(c) 1-8% by weight crosslinked polyvinylpyrrolidone,
(d) 0.1-2% by weight magnesium stearate,
(e) 0.1-1% by weight colloidal silicon dioxide, and
   the external phase comprising:
(f) 0.1-2% by weight magnesium stearate,
(g) 0.1-1% by weight colloidal silicon dioxide, and
   based on the total weight of the content of the capsule.

A capsule for oral administration comprising an inner phase and an external phase, the inner phase comprising:
(a) 3-62% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], present as fumarate salt,
(b) 20-85% by weight of lactose and cellulose,
(c) 1-8% by weight crosslinked polyvinylpyrrolidone,
(d) 0.1-1% by weight magnesium stearate,
(e) 0.1-1% by weight colloidal silicon dioxide, and
   the external phase comprising:
(f) 0.1-1% by weight magnesium stearate,
(g) 0.1-1% by weight colloidal silicon dioxide, and Optionally,
(a) 1-2% by weight crosslinked polyvinylpyrrolidone,
(b) 1-10% by weight of lactose and cellulose,
   based on the total weight of the content of the capsule.

A capsule for oral administration comprising, consisting essentially of or consisting of:
(a) 10-20% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], present as fumarate salt,
(b) 70-85% by weight of lactose and cellulose,
(c) 3-8% by weight crosslinked polyvinylpyrrolidone,
(d) 0.5-1.5% by weight magnesium stearate, and
(e) 0.25-1% by weight colloidal silicon dioxide,
   based on the total weight of the content of the capsule.

A capsule for oral administration comprising, consisting essentially of or consisting of:
(a) 30-62% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], present as fumarate salt,
(b) 25-40% by weight of lactose and cellulose,
(c) 5-8% by weight crosslinked polyvinylpyrrolidone,
(d) 0.5-1.5% by weight magnesium stearate, and
(e) 0.5-1% by weight colloidal silicon dioxide,
   based on the total weight of the content of the capsule.

A capsule for oral administration comprising, consisting essentially of or consisting of:
(a) 50-56% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], present as fumarate salt,
(b) 20-40% by weight of lactose and cellulose,
(c) 5-8% by weight crosslinked polyvinylpyrrolidone,
(d) 0.1-1.5% by weight magnesium stearate, and
(e) 0.5-1% by weight colloidal silicon dioxide,
   based on the total weight of the content of the capsule.

### Definitions

The term “pharmaceutically acceptable salts” refers to salts that can be formed, for example, as acid addition salts, preferably with organic or inorganic acids. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. The term “pharmaceutically acceptable” refers to those compounds, materials, compositions, and/or dosage forms which are suitable for use in contact with the tissues of human beings and animals without excessive toxicity, irritation, allergic response, other problem or complication, commensurate with a reasonable benefit/risk ratio.

The term “treat”, treating” or “treatment” of any disease or disorder refers to ameliorating the disease or disorder (e.g. slowing, arresting or reducing the development of the disease, or at least one of the clinical symptoms thereof), to preventing, or delaying the onset, or development, or progression of the disease or disorder. In addition those terms refer to alleviating or ameliorating at least one physical parameter including those which may not be discernible by the patient and also to modulating the disease or disorder, either physically (e.g. stabilization of a discernible symptom), physiologically (e.g. stabilization of a physical parameter), or both.

The term “about”, as used herein, is intended to provide flexibility to a numerical range endpoint, providing that a given value may be “a little above” or “a little below” the endpoint accounting for variations one might see in the measurements taken among different instruments, samples, and sample preparations. The term usually means within 10%, preferably within 5%, and more preferably within 1% of a given value or range.

The terms “pharmaceutical composition” or “formulation” can be used herein interchangeably, and relate to a physical mixture containing a therapeutic compound to be administered to a mammal, e.g. a human, in order to prevent, treat, or control a particular disease or condition affecting a mammal. The terms also encompass, for example, an intimate physical mixture formed at high temperature and pressure.

The term “oral administration” represents any method of administration in which a therapeutic compound can be administered through the oral route by swallowing, chewing, or sucking an oral dosage form. Such oral dosage forms are traditionally intended to substantially release and/or deliver the active agent in the gastrointestinal tract beyond the mouth and/or buccal cavity.

The term “a therapeutically effective amount” of a compound, as used herein, refers to an amount that will elicit the biological or medical response of a subject, for example, ameliorate symptoms, alleviate conditions, slow or delay disease progression, etc. The term “a therapeutically effective amount” also refers to an amount of the compound that, when administered to a subject, is effective to at least partially alleviate and/or ameliorate a condition, a disorder, or a disease. The term “effective amount” means the amount of the subject compound that will engender a biological or medical response in a cell, tissue, organs, system, animal or human that is being sought by the researcher, medical doctor or other clinician.

The term “comprising” is used herein in its open ended and non-limiting sense unless otherwise noted. In a more limited embodiment “comprising” can be replaced by “consisting of”, or “consisting essentially of” which is no longer open-ended. In a most limited version it can include only feature steps, or values as listed in the respective embodiment.

### EXAMPLES

The following examples illustrate the invention and provide support for the disclosure of the present invention without limiting the scope of the invention.

### Example 1: Physiochemical Properties of Compound A

The fumarate salt of Compound A exhibits a pH dependent solubility profile as provided in Table 1. The fumarate shows a solubility of greater or equal to 2 mg/mL in pH 1.2 to pH 4.7, 0.03 mg/ml at pH 6.8 buffer. Solubility of the fumarate in FaSSIF is 0.09 mg/mL and 0.21 mg/mL in FeSSIF. However, at pH values of 5.4 and above, the solid residues obtained after 24h equilibration correspond to the free form, as its solubility is lower than that of the fumarate in this pH range. The fumarate salt is non hygroscopic. The maximum water uptake by DVS is less than 0.1% up to 95% RH at 25° C.

TABLE 1
Solubility of Compound A fumarate at 25° C.

| Solvent | Solubility (mg/mL) |
|---|---|
| Water | >2 |
| Ethanol | >100 |
| Acetonitrile | >100 |
| pH 1.2 buffer (hydrochloric acid) | >2 |
| pH 2 buffer (hydrochloric acid) | >2 |
| pH 4.7 buffer (acetate) | >2 |
| pH 6.8 buffer (phosphate) | 0.03 |
| pH 2.0 (SGF) ^{a)} | >2 |
| pH 6.5 (FaSSIF) ^{b)} | 0.09 |
| pH 5.8 (FeSSIF) ^{c)} | 0.21 |

^{a)} Simulated gastric fluid
^{b)} Fasted state simulating intestinal fluid
^{c)} Fed state simulating intestinal fluid

### Example 2: Excipient Compatibility

Compound A fumarate API compatibility was performed with excipient mixtures and capsules powders according to compositions described in Table 2. The test conditions are presented in Table 3 along with the observed degradation percent of the excipient mixtures and capsules powders.

TABLE 2
Compositions of the excipient mixtures [mass-%]

| | | |
|---|---|---|
| No. | Material | % w/w |

Mixture 1 - Dry Blend

| | | |
|---|---|---|
| 1 | Mannitol powder | 47.4 |
| 2 | Starch 1500 | 36.8 |
| 3 | Sodium starch glycolate | 6.3 |
| 4 | Hydroxypropyl cellulose 100 cps | 4.2 |
| 5 | Sodium stearyl fumarate | 3.2 |
| 6 | Talc | 2.1 |

Mixture 2 - Wet granulation

| | | |
|---|---|---|
| 1 | MCC PH101 | 29.5 |
| 2 | Lactose monohydrate | 56.8 |
| 3 | Croscarmellose sodium | 6.3 |
| 4 | Hydroxypropyl methylcellulose 3 cps | 4.2 |
| 5 | Magnesium stearate | 1.1 |
| 6 | AEROSIL ® 200 | 2.1 |

Mixture 3 - Roller compaction

| | | |
|---|---|---|
| 1 | MCC PH102 | 55.8 |
| 2 | Calcium hydrogen phosphate | 22.1 |
| 3 | L-Hydroxypropyl cellulose | 10.5 |
| 4 | Hydroxypropyl cellulose 100 cps | 5.3 |
| 5 | Magnesium stearate | 3.2 |
| 6 | AEROSIL ® 200 | 3.2 |

TABLE 3
Compatibilities of Compound A fumarate capsule/excipients mixes

| | Temp [° C.] and | DP [%] |
|---|---|---|
| | Humidity [%], | fumarate |
| Capsule/excipients mix | duration | P90 |
| 1% API in mixture 1 | 50° C., tight | 0.42 |
| 1% API in mixture 2 | container, 2 wks | 0.43 |
| 1% API in mixture 3 | | 0.74 |
| HPMC capsule powder | | 1.01 |
| HGC capsule powder | | 0.45 |
| 1% API in mixture 1 | 50° C./75% RH/2 | 0.46 |
| 1% API in mixture 2 | wks | 0.53 |
| 1% API in mixture 3 | | 0.49 |
| HPMC capsule powder | | 1.04 |
| HGC capsule powder | | 0.44 |

In bulk, Compound A fumarate was stable under all tested conditions, with the exception of HMPC capsule powder, where degradation >1% was observed at 50° C. in tight container as well as at 50° C. and 75% RH after 2 weeks storage.

### Example 3: Accelerated Stability Assessment Program (ASAP) Study

The compatibility of Compound A fumarate was further evaluated in 21 days ASAP study involving three different formulation principles, wet granulation, dry blending and roller compaction. The composition details of the blends are presented in Table 4. The drug load selected for study was 5% w/w, translating to 6.40% w/w of fumarate salt. The XRPD of the samples were evaluated at certain time intervals according to the Safety Protocol in Table 5.

Dry blend compositions were prepared by manual sieving followed by mixing in TURBULA® mixer. Wet granulation was performed manually; with water uptake of 30% w/w. Roller compaction was performed with press force of 5 KN/cm, roll gap of 1 mm and roll speed of 2 RPM. The size of milling screen used was 0.80 mm.

TABLE 4
Compositions tested in ASAP study

| | P4 | P5 | P6 | P7 | P8 |
|---|---|---|---|---|---|
| | Dry | Dry | Wet | Roller | Roller |
| Ingredients | blend | blend | granulation | compaction | compaction |
| Internal Phase | | | | | |
| Compound A Fumarate | 6.40 | 6.40 | 6.40 | 6.40 | 6.40 |
| Mannit PH | — | — | 58.58 | — | — |
| Lactose, spray dried | 58.66 | — | — | — | — |
| AVICEL ® PH 101 | — | — | 23.02 | — | — |
| Cellulose MK GR | 27.94 | 27.94 | — | 81.60 | 34.88 |
| Mannitol DC | — | 58.66 | — | — | — |
| Lactose, Anhydrous | — | — | — | — | 36.72 |
| PVP-K 30 | — | — | 5.00 | — | — |
| Polyvinyl pyrrolidone XL | 5.00 | | 5.00 | — | — |
| Sodium starch glycolate | — | — | — | — | — |
| Crosscarmellose sodium | — | 5.00 | — | — | — |
| Sodium stearyl fumarate | — | — | — | — | — |
| AEROSIL ® 200 PH | — | — | — | 0.25 | 0.25 |
| Magnesium stearate | | | | 0.50 | 0.50 |
| External Phase | | | | | |
| Cellulose MK GR | — | — | — | — | 15.00 |
| Sodium starch glycolate | — | — | — | — | 2.00 |
| Sodium stearyl fumarate | — | — | 1.50 | — | — |
| AEROSIL ® 200 PH | 0.50 | 0.50 | 0.50 | 0.25 | 0.25 |
| Magnesium stearate | 1.50 | 1.50 | — | 1.00 | 1.00 |
| Total | 100.00 | 100.00 | 100.00 | 100.00 | 100.00 |
| Batch Size (g) | 50.00 | 50.00 | 40.00 | 100.00 | 100.00 |

TABLE 5
ASAP stability protocol details

| | | Days | | | | |
|---|---|---|---|---|---|---|
| T (° C.) | % RH | 0 | 3 | 7 | 12 | 21 |
| 5 | — | x | — | — | — | — |
| 50 | 75 | — | — | xy | — | xy |
| 60 | 41 | — | — | — | — | x |
| 60 | 80 | — | — | xy | x | xxy |
| 70 | 11 | — | — | — | — | x |
| 70 | 41 | — | — | y | xx | xy |
| 70 | 75 | — | x | x | x | Z |
| 80 | 11 | — | — | x | x | — |
| 80 | 50 | — | x | xx | — | y |

x: indicate sample time point for assay and degradation product (DP)
y: indicate sample time point for XRPD

### Example 4: Variant Selection

The results of ASAP analysis are presented in Table 6, Table 7 and Table 8. ASAP analysis was confined only to major degradation products observed in all five compositions. They were identified at relative retention time (RRT) of 0.53 min, 0.64 min and 0.96 min in the chromatogram. Shelf life prediction was done by considering size 1 HGC capsule and 120cc/30 count HDPE bottle without desiccant packaging.

At 25° C./60% RH and 5° C. the shelf life was predicted for 3 years with 100% probability to pass for all five compositions. Based on probability to pass at 40° C./75% RH, P4 (dry blend) and P7 (roller compaction) appeared to be most stable compositions.

TABLE 6
ASAP assessment for degradation product at RRT 0.53 min
Degradation product at RRT 0.53 min

| | | | | | | | P8 | |
|---|---|---|---|---|---|---|---|---|
| | | | | | | | Roller | |
| | | P4 | P5 | | P6 | P7 | compaction | |
| | | Dry | Dry | | Wet | Roller | Without | |
| | | blend | blend | | granulation | compaction | 12D*70/75, | |
| Parameters | | With all data | With all data | | With all data | With all data | 21D*60/80 | |
| 5° C. | Shelf life | 3 | 3 | | 3 | 3 | 3 | |
| | evaluated for | | | | | | | |
| | period | | | | | | | |
| | (years) | | | | | | | |
| | % Probability | 100 | 100 | | 100 | 100 | 100 | |
| | to pass for | | | | | | | |
| | that period | | | | | | | |
| 25° C./60% | Shelf life | 3 | 3 | | 3 | 3 | 3 | |
| RH | evaluated for | | | | | | | |
| | period | | | | | | | |
| | (years) | | | | | | | |
| | % Probability | 100 | 100 | | 100 | 100 | 100 | |
| | to pass for | | | | | | | |
| | that period | | | | | | | |
| 30° C./75% | Shelf life | 3 | 3 | | 3 | 3 | 3 | |
| RH | evaluated for | | | | | | | |
| | period | | | | | | | |
| | (years) | | | | | | | |
| | % Probability | 100 | 97 | | 100 | 100 | 96 | |
| | to pass for | | | | | | | |
| | that period | | | | | | | |
| 40° C./75% | Shelf life | 3 | 3 | 1.5 | 3 | 3 | 3 | 1.5 |
| RH | evaluated for | | | | | | | |
| | period | | | | | | | |
| | (years) | | | | | | | |
| | % Probability | 100 | 64 | 92 | 100 | 86 | 49 | 85 |

to pass for
that period

*D stands for days

TABLE 7
ASAP assessment for degradation product at RRT 0.64 min
Degradation Product at RRT 0.64

| | | | | | P7 | | |
|---|---|---|---|---|---|---|---|
| | | | | | Roller | | |
| | | | | P6 | compaction | P8 | |
| | | P4 | | Wet | Without | (RRT 0.66) | |
| | | Dry | P5 | granulation | 7D*50/75, | Roller | |
| | | blend | Dry | Without | 12D*70/75, | compaction | |
| Parameters | | With all data | blend | 70/75, 60/80 | 21D*60/80 | With all data | |
| 5° C. | Shelf life | 3 | Throughout | 3 | 3 | 3 | |
| | evaluated for | | the study it | | | | |
| | period | | was | | | | |
| | (years) | | observed | | | | |
| | % Probability | 100 | 0.02% for all | 100 | 100 | 100 | |
| | to pass for | | the storage | | | | |
| | that period | | conditions | | | | |
| 25° C./60% | Shelf life | 3 | | 3 | 3 | 3 | |
| RH | evaluated for | | | | | | |
| | period | | | | | | |
| | (years) | | | | | | |
| | % Probability | 100 | | 100 | 100 | 98 | |
| | to pass for | | | | | | |
| | that period | | | | | | |
| 30° C./75% | Shelf life | 3 | | 3 | 3 | 3 | |
| RH | evaluated for | | | | | | |
| | period | | | | | | |
| | (years) | | | | | | |
| | % Probability | 100 | | 100 | 100 | 87 | |
| | to pass for | | | | | | |
| | that period | | | | | | |
| 40° C./75% | Shelf life | 3 | | 3 | 3 | 3 | 1.5 |
| RH | evaluated for | | | | | | |
| | period | | | | | | |
| | % Probability | 100 | | 88 | 100 | 47 | 82 |

to pass for
that period

*D stands for days

TABLE 8
ASAP assessment for degradation product at RRT 0.96 min
Degradation Product at RRT 0.96

| | | P4 | | P5 | | P6 | P7 | | P8 | |
|---|---|---|---|---|---|---|---|---|---|---|
| | | Dry | | Dry | | Wet | Roller | | Roller | |
| | | blend | | blend | | granulation | compaction | | compaction | |
| Parameter | | With all data | | With all data | | With all data | With all data | | With all data | |
| 5° C. | Shelf life | 3 | | 3 | | 3 | 3 | | 3 | |
| | evaluated for | | | | | | | | | |
| | period | | | | | | | | | |
| | (years) | | | | | | | | | |
| | % Probability | 100 | | 100 | | 100 | 100 | | 100 | |
| | to pass for | | | | | | | | | |
| | that period | | | | | | | | | |
| 25° C./60% | Shelf life | 3 | | 3 | | 3 | 3 | | 3 | |
| RH | evaluated for | | | | | | | | | |
| | period | | | | | | | | | |
| | (years) | | | | | | | | | |
| | % Probability | 100 | | 100 | | 100 | 100 | | 100 | |
| | to pass for | | | | | | | | | |
| | that period | | | | | | | | | |
| 30° C./75% | Shelf life | 3 | | 3 | | 3 | 3 | | 3 | |
| RH | evaluated for | | | | | | | | | |
| | period | | | | | | | | | |
| | (years) | | | | | | | | | |
| | % Probability | 99 | | 63 | | 100 | 100 | | 96 | |
| | to pass for | | | | | | | | | |
| | that period | | | | | | | | | |
| 40° C./75% | Shelf life | 3 | 1 | 3 | 1 | 3 | 3 | 1 | 3 | 1 |
| RH | evaluated for | | | | | | | | | |
| | period | | | | | | | | | |
| | (years) | | | | | | | | | |
| | % Probability | 41 | 93 | 0 | 13 | 93 | 27 | 100 | 1 | 91 |

to pass for
that period

The results of disproportionation are summarized in Table 9. The analysis was done for the samples stored at 21 days. Disproportionation to the base was noted at all selected conditions for wet granulated sample P6 whereas batch P4 prepared by direct blending did not show any disproportionation. For roller compacted batches, composition of batch P8 was found to be more stable for disproportionation as, it was observed only at condition of 80° C./50% RH compared to P7 composition. Based on ASAP results and disproportionation, P4 was found to be the most stable composition.

TABLE 9
Disproportionation results for ASAP batches

| | Stability conditions | | | |
|---|---|---|---|---|
| | 50° C./75% RH −21 | 60° C./80% RH −21 | 70° C./41% RH −21 | 80° C./50% RH −21 |
| Batch Numbers | days | days | days | days |
| Compound A DS | No free form peak | No free form peak | No free form peak | No free form peak |
| | around 9.7°2θ | around 9.7°2θ | around 9.7°2θ | around 9.7°2θ |
| P4 | No free form peak | No free form peak | No free form peak | No free form peak |
| | around 9.7°2θ | around 9.7°2θ | around 9.7°2θ | around 9.7°2θ |
| P5 | No free form peak | Small peak of free | Small peak of free | Small peak of free |
| | around 9.7°2θ | form around 9.7°2θ | form around 9.7°2θ | form around 9.7°2θ |
| P6 | Small peak of free | Small peak of free | Small peak of free | Small peak of NXA |
| | form around 9.7°2θ | form around 9.7°2θ | form around 9.7°2θ | around 9.7°2θ |
| P7 | No free form peak | Small peak of NXA | No sample | Small peak of free |
| | around 9.7°2θ | around 9.7°2θ | available | form around 9.7°2θ |
| P8 | No free form peak | No free form peak | No NXA free form | Small peak of free |
| | around 9.7°2θ | around 9.7°2θ | around 9.7°2θ | form around 9.7°2θ |

### Example 4: Composition Optimization

Two compositions of 150 mg (batch numbers P13 and P14) were evaluated to determine the impact of roller compaction (RC) on bulk density and tapped density of the blend and dissolution profile as provided in Table 10.

TABLE 10
Compositions of Compound A fumarate 150 mg strength

| | P13 | | P14 | |
|---|---|---|---|---|
| Components | mg/unit | % w/w | mg/Unit | % w/w |

Inner Phase

| | | | | |
|---|---|---|---|---|
| Compound A Fumarate | 189.45 | 61.11 | 189.45 | 61.11 |
| Lactose, spray dried | — | — | 64.86 | 20.92 |
| Cellulose MK GR | 98.84 | 31.88 | 33.24 | 10.72 |
| Polyvinyl | 15.50 | 5.00 | 15.50 | 5.00 |
| polypyrrolidone XL | | | | |
| AEROSIL ® 200 PH | 0.78 | 0.25 | 1.55 | 0.50 |
| Magnesium stearate | 1.55 | 0.50 | 3.10 | 1.00 |
| AEROSIL ®200 PH | 0.78 | 0.25 | 0.75 | 0.24 |
| Magnesium stearate | 3.10 | 1.00 | 1.55 | 0.50 |
| Total | 310.00 | 100.00 | 310.00 | 100.00 |
| Capsule Shell | Size 0 HGC | | Size 0 HGC | |

The feasibility of filing of pre-RC blend and RC blend for 150 mg strength with P13 was evaluated by manual filling of blend in size “0” hard gelatin capsule (HGC) as provided in Table 11.

TABLE 11
Impact of roller compaction on bulk density and tapped density

| Parameters | Pre-RC blend of P13 | RC granules of P13 |
|---|---|---|
| Bulk density (g/mL) | 0.500 | 0.63 |
| Tapped density (g/mL) | 0.769 | 0.90 |
| Amount filled manually in size “0” | 315.23 | N.A. |
| capsules (mg) | | |
| Observations on fill | 90% filled in capsule body | N.A. |

Roller compaction improved density of blend which in turn could allow scope for higher fill weight achievement for 150 mg strength in size “0” capsules.

The dissolution profile of P14, adapted from ASAP composition P4, was evaluated with dissolution media of 0.1N and 0.01N hydrochloric acid as provided in Table 12. In 0.1N hydrochloric acid, almost 95% drug was released at the end of 10 minutes. For better discrimination on formulation or process changes; dissolution was carried out in 0.01N hydrochloric acid. Dissolution was found to be slower with 57.22% drug released at the end of 30 minutes.

TABLE 12
Comparative dissolution profile of
Compound A fumarate 150 mg strength

| | B. No. P23* | |
|---|---|---|
| | % released | % released |
| Time (min) | (0.1N hydrochloric acid) | (0.01N hydrochloric acid) |
| 5 | 49.96 | 3.37 |
| 10 | 95.38 | 12.90 |
| 15 | 100.85 | 25.33 |
| 30 | 101.50 | 57.22 |
| 60 | 101.50 | 91.38 |

Dissolution conditions: paddle with sinkers at 50 RPM and 900 mL media volume
*Composition identical to P14

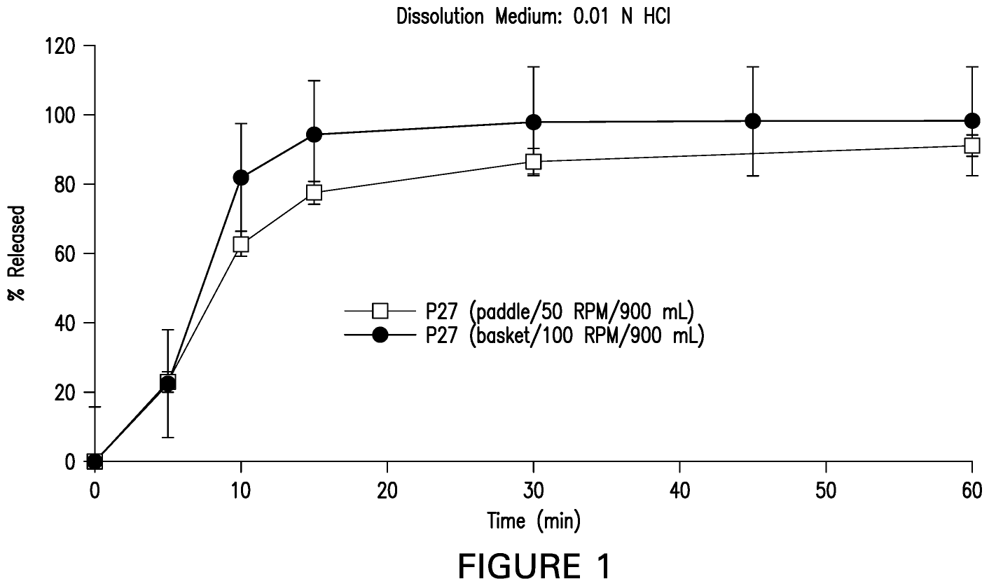

Physical observation of capsules during dissolution indicated formation of soft agglomerates entrapped within sinkers, which appeared to be dissolving slowly resulting in low release at 60 minutes. Absence of swelling components in extra-granular phase could have attributed to lag in initiating capsule disintegration and low release. Composition of P14 was further modified to include extra-granular components as provided in Table 13 and tested for dissolution in 0.01 N hydrochloric acid in depicted in FIG. 1 and Table 14.

TABLE 13
Modified composition of Compound A fumarate 150 mg strength

P27

| Ingredients | mg/Unit | % w/v |
|---|---|---|
| Inner Phase | | |
| Compound A fumarate | 189.45 | 54.13 |
| Lactose, spray dried | 64.86 | 18.53 |
| Cellulose MK GR | 33.24 | 9.50 |
| Polyvinyl polypyrrolidone XL | 15.50 | 4.43 |
| AEROSIL ® 200 PH | 1.55 | 0.44 |
| Magnesium stearate | 3.10 | 0.89 |
| External phase | | |
| Cellulose MK GR | 22.53 | 6.44 |
| Lactose, spray dried | 11.55 | 3.30 |
| Polyvinyl polypyrrolidone XL | 5.38 | 1.54 |
| AEROSIL ® 200 PH | 1.29 | 0.37 |
| Magnesium stearate | 1.55 | 0.44 |
| Total | 350.00 | 100.00 |

| | |
|---|---|
| Capsule Shell | Size 0 HGC |

TABLE 14
Dissolution data of modified composition of 150
mg strength in 900 ml of 0.01N hydrochloric acid

| | P27 | | | |
|---|---|---|---|---|
| | Paddle with sinkers at 100 RPM | | Basket at 100 RPM | |
| | % Released | | % Released | |
| Time (min) | (n = 3) | SD | (n = 3) | SD |
| 5 | 23.08 | 2.93 | 22.51 | 4.41 |
| 10 | 62.67 | 3.60 | 81.93 | 2.53 |
| 15 | 77.52 | 3.19 | 94.18 | 1.90 |
| 30 | 86.64 | 3.52 | 98.04 | 0.13 |
| 45 | 91.07 | 3.04 | 98.08 | 0.07 |
| 60 | 98.96 | 0.95 | 98.07 | 0.27 |

The dissolution for the P27 composition was carried out at 100 RPM with USP-I/basket and at 50 RPM using USP-II/paddle with sinker. The addition of extra-granular components promoted faster opening and disintegration of capsules resulting in improved dissolution profile compared to P23 batch.

In case of paddle with sinker, accumulation of powder was observed beneath the sinker at the end of 60 minutes; indicating cone effect. With basket, complete dispersion of contents were obtained with no accumulation of powder and dissolution was also found to be faster. Basis these observations, 0.01 N hydrochloric acid with basket/USP-1 apparatus operated at 100 RPM was finalized as dissolution method for Compound A capsules.

### Example 5: Dissolution Optimization

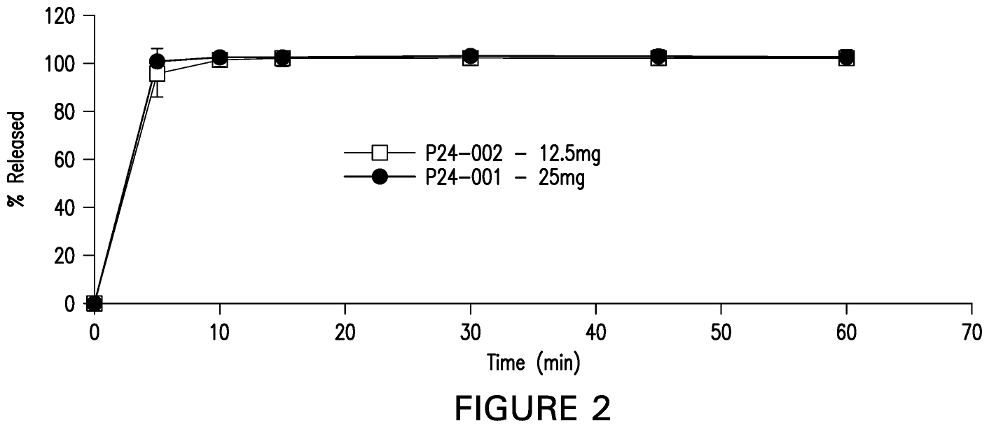

Compositions of 12.5 mg and 25 mg were adapted from P4 of the ASAP study and prepared as a common blend, filled in size 1 capsules for 25 mg and size 2 capsules for 12.5 mg, as provided in Table 15. Dissolution of these variants in final dissolution medium (0.01N hydrochloric acid with basket/USP-1 apparatus/900 mL) is captured in the FIG. 2 and Table 16.

TABLE 15
Composition of Compound A fumarate capsules
25 mg and 12.5 mg strengths

| | | | P24-002 |
|---|---|---|---|
| | | | (12.5 mg) & |
| | Composition | P24-001 | P17 |
| | (% w/w) | (25 mg) | (12.5 mg)* |
| Ingredients | % w/w | mg/Unit | mg/Unit |
| Inner phase | | | |
| Compound A | 15.79 | 31.58 | 15.79 |
| fumarate | | | |
| Lactose, | 52.71 | 105.43 | 52.71 |
| spray dried | | | |
| Cellulose MK GR | 25.00 | 50.00 | 25.00 |
| Polyvinyl | 5.00 | 10.00 | 5.00 |
| polypyrrolidone XL | | | |
| AEROSIL ® 200 PH | 0.25 | 0.50 | 0.25 |
| Magnesium stearate | 0.50 | 1.00 | 0.50 |
| External phase | | | |
| AEROSIL ® 200 PH | 0.25 | 0.50 | 0.25 |
| Magnesium stearate | 0.50 | 1.00 | 0.50 |
| Total | 100.00 | 200.00 | 100.00 |
| Capsule shell | | HGC size “1” | |
| Capsule shell | | | HGC size “2” |

TABLE 16
Dissolution data for compositions in 0.01N hydrochloric acid

| | P24-001 (25 mg) | | P24-002 (12.5 mg) | |
|---|---|---|---|---|
| | % Released | | % Released | |
| Time (min) | (n = 6) | SD | (n = 6) | SD |
| 5 | 100.64 | 0.82 | 95.67 | 10.14 |
| 10 | 102.37 | 1.33 | 100.99 | 2.66 |
| 15 | 102.41 | 1.35 | 101.54 | 3.05 |
| 30 | 102.57 | 1.29 | 101.79 | 2.98 |
| 45 | 102.86 | 1.21 | 101.95 | 3.22 |
| 60 | 102.43 | 1.07 | 101.93 | 3.02 |

Dissolution conditions: Basket at 100 RPM and 900 mL media volume

### Example 6: Microenvironment pH Determination

To assess the potential risk of disproportionation, the pH of blends P17 (based on P24-002), P27, and P32 (based on P27), was recorded to assess probability of disproportionation. The contents of the capsule was dispersed in 5 mL of milli Q water. The resulting dispersion was vortexed for a period of 5 minutes and pH was recorded. The pH of the blend of final formulation was found to be below 4.00 as provided in Table 17.

TABLE 17
Microenvironment pH of blends of Compound A

| Sr. No. | Batch Number | Strength | | pH |
|---|---|---|---|---|
| 1 | P17* | 12.5 | mg | 3.76 |
| 2 | P27 | 150 | mg | 3.79 |
| 3 | P32** | 100 | mg | 3.75 |

*Composition similar to batch P24-002 (Table 14)
**Composition similar to P27; filled equivalent to 100 mg fill weight (Table 12)

### Example 7: Development Stability Study

In parallel to dissolution medium optimization, a four week development stability study was initiated for compositions of 12.5 mg and 150 mg. The compositions selected for this study were identical to batch P14 for 150 mg strength (Table 10) and P17 (Table 15) for 12.5 mg strength. The formulations were filled in both HGC as well as HPMC capsules. The stability plan is depicted in Table 18.

TABLE 18
Stability conditions and packaging for development stability

| Condition | Packaging |
|---|---|
| 25° C./60% RH | Open petri dish |
| 25° C./60% RH | HDPE bottles (90 cc) with CRC cap* |
| 40° C./75% RH | Open petri dish |
| 40° C./75% RH | HDPE bottles (90 cc) with CRC cap* |

*Test performed in case discrepancy observed in open petri dish

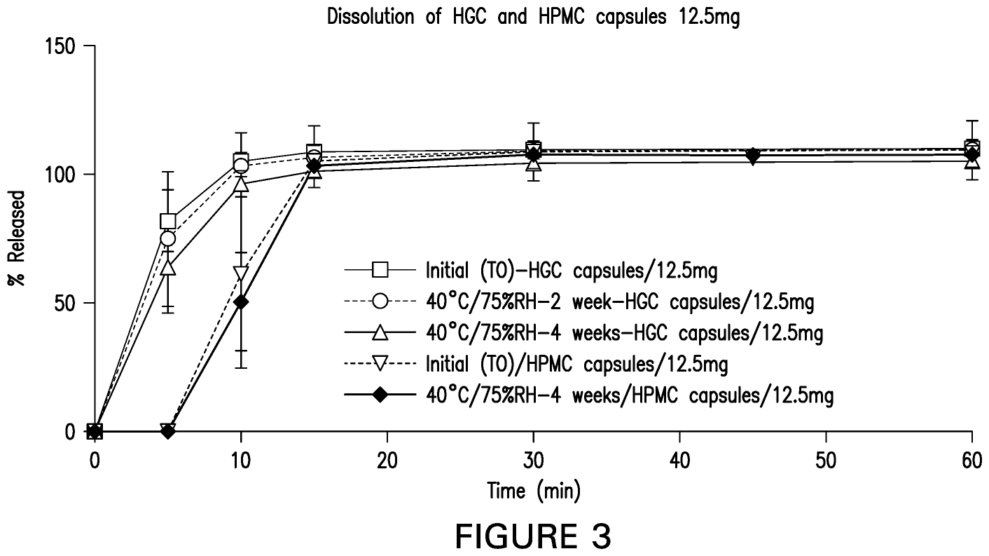

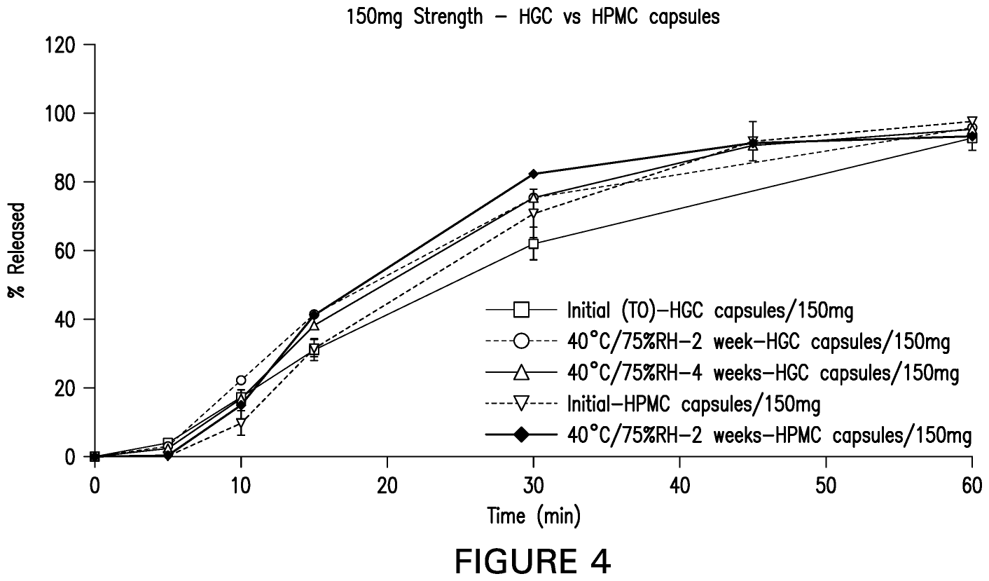

The dissolution of stability samples were carried out in 900 mL of 0.1 N Hydrochloric acid using paddles with sinker. The comparative dissolution profiles of 12.5 mg and 100 mg strengths are presented in FIG. 3 and FIG. 4 respectively. Lag time of around 10 minutes was observed for dissolution of HPMC capsules compared to HGC capsules. No significant difference in dissolution from initial was observed for HGC and HPMC capsules for both the strengths.

No significant change from initial was observed for assay and degradation products for capsules exposed in open petri-plate condition. All the individual impurities were found to be less than 0.2% and total impurities were found to be below 0.5% w/w.

An XRPD analysis was carried to evaluate disproportionation if any of Compound A fumarate salt over to base. The samples were analyzed at 4 weeks. No change in XRPD pattern was observed for HGC and HPMC capsules stored at 25° C./60% RH in open petri-plate and HDPE bottles. However, early signs of disproportionation was observed in both capsule types stored at 40° C./75% RH.

Stability studies revealed comparable product characteristics for both HGC and HPMC capsules. HPMC capsules were selected for clinical batches as they have lower moisture content than HGC capsules. Moisture could be one of the trigger for initiating disproportionation. However, HPMC capsules may require the use of a desiccant, which may provide protection against disproportionation for long term stability.

### Example 8: Technical Stability

Compositions of 12.5 mg, 25 mg, and 100 mg strengths of Compound A were evaluated for technical stability in both hard gelatin (HGC) and hypromellose-based (HPMC) capsules as provided in Tables 19 and 20.

TABLE 19
Compositions of 12.5 mg and 25 mg strengths of Compound A

| | | Composition | Composition | |
|---|---|---|---|---|
| | Composition | per unit [mg] | per unit [mg] | |
| Ingredient | per unit [%] | 12.5 mg* | 25 mg** | Function |
| Inner Phase | | | | |
| Compound A fumarate | 15.90 | 15.90*** | 31.80*** | Drug substance |
| Lactose spray-dried | 52.71 | 52.71 | 105.20 | Diluent |
| Cellulose MK GR | 25.00 | 25.00 | 50.00 | Diluent |
| Polyvinyl | 5.00 | 5.00 | 10.00 | Disintegrant |
| polypyrrolidone XL | | | | |
| AEROSIL ® 200 PH | 0.25 | 0.25 | 0.50 | Glidant |
| Magnesium stearate | 0.50 | 0.50 | 1.00 | Lubricant |
| Subtotal of inner blend | | 99.25 | 99.25 | 198.50 |
| External phase | | | | |
| AEROSIL ® 200 PH | 0.25 | 0.25 | 0.50 | Glidant |
| Magnesium stearate | 0.50 | 0.50 | 1.00 | Lubricant |
| HGC/HPMC (size 2/3) | — | 48.00 | | Capsule shell |
| HGC/HPMC (size 1) | — | | 76.00 | Capsule shell |
| Total weight | | 100.00 | 148.00 | 276.00 |

*B. No. P33/P34
**B. No. P35/P36
***The quantity of drug substance (DS) is corrected for salt factor (1.263) and for assay (99.3%).
The compensation for assay correction is done by adjusting the quantity of lactose spray-dried.

TABLE 20
Compositions of 100 mg strength of Compound A

| | Composition | Composition | |
|---|---|---|---|
| | per unit | per unit | |
| Ingredient | [%] | [mg]* | Function |
| Inner phase | | | |
| Compound A fumarate | 55.30 | 127.19** | Drug substance |
| Lactose spray-dried | 17.45 | 40.14 | Diluent |
| Cellulose MK GR | 9.15 | 21.03 | Diluent |
| Polyvinyl | 4.49 | 10.33 | Disintegrant |
| polypyrrolidone XL | | | |
| AEROSIL ® 200 PH | 0.45 | 1.03 | Glidant |
| Magnesium stearate pharma | 0.90 | 2.07 | Lubricant |
| Subtotal of Inner blend | | 201.80 | |
| External Phase | | | |
| Cellulose MK GR | 6.53 | 15.02 | Diluent |
| Lactose spray dried | 3.35 | 7.70 | Diluent |
| Polyvinyl | 1.56 | 3.50 | Disintegrant |
| polypyrrolidone XL | | | |
| AEROSIL ® 200 PH | 0.37 | 0.86 | Glidant |
| Magnesium | 0.45 | 1.03 | Lubricant |
| stearate pharma | | | |
| HGC/HPMC (size 0) | | 96.000 | Capsule Shell |
| Total weight | | 100.00 | 326.00 |

*B. No. P37/P38
**The quantity of drug substance (DS) is corrected for salt factor (1.263) and for assay (99.3%).
The compensation for assay correction is done by adjusting the quantity of lactose spray-dried.

### Roller Compaction Evaluation

The effect of roll pressure on dissolution was evaluated for blends filled in HPMC capsules and for compositions of 12.5 mg and 100 mg strengths according to table 18 and 19, respectively, to determine operating range of roll pressure during compaction while using qualified range of roll gap as 2 mm for manufacturing.

The dissolution was carried out in 0.01 N hydrochloric acid with basket/USP-1 apparatus operated at 100 RPM and 900 mL media volume. Dissolution profiles of batches for both 12.5 mg and 100 mg strength were found to be comparable across the roll pressure range studied. High variability in release was noted until 15 minutes, which could be attributed to variability in opening of HPMC capsules. Nevertheless, >90% drug release was observed for at the end of 15 minutes for 12.5 mg strength. For 100 mg strength, complete drug release was achieved at the end of 30 minutes.

### Example 9: Hard Cellulose-Based Capsule

Manufacturing formula for a 12.5 mg, 25 mg and 100 mg hard, cellulose based capsules of Compound A

| | Amount per batch (g) | | |
|---|---|---|---|
| Ingredient | 12.5 mg | 25 mg | 100 mg |
| Inner Phase/Dry Mix | | | |
| Compound A Fumarate (salt) ¹ | 157.900 (125.000) | 315.800 (250.000) | 1263.000 (1000.000) |
| (corresponding to DS base) | | | |
| Lactose Spray-dried | 527.100 | 1054.200 | 410.400 |
| Cellulose MK GR | 250.000 | 500.000 | 210.300 |
| Polyvinyl pyrrolidone XL | 50.000 | 100.000 | 103.300 |
| AEROSIL ® 200 PH | 2.500 | 5.000 | 10.300 |
| Magnesium Stearate Pharma | 5.000 | 10.000 | 20.700 |
| Outer Phase | | | |
| Cellulose MK GR | — | — | 150.200 |
| Lactose Spray-dried | — | — | 77.000 |
| Polyvinyl pyrrolidone XL | — | — | 35.900 |
| AEROSIL ® 200 PH | 2.500 | 5.000 | 8.600 |
| Magnesium Stearate Pharma | 5.000 | 10.000 | 10.300 |
| Weight capsule fill mix | 1000.000 | 2000.000 | 2300.000 |
| Empty capsule shell, | | | |
| (theoretical weight) | | | |
| HCC size 3 | 480.000 | — | — |
| HCC size 1 | — | 760.000 | — |
| HCC size 0 | — | — | 960.000 |
| Total batch weight | 1480.000 | 2760.000 | 3260.000 |

### Example 9A: Manufacturing of 12.5 mg and 25 mg Hard, Cellulose Capsules of Compound A

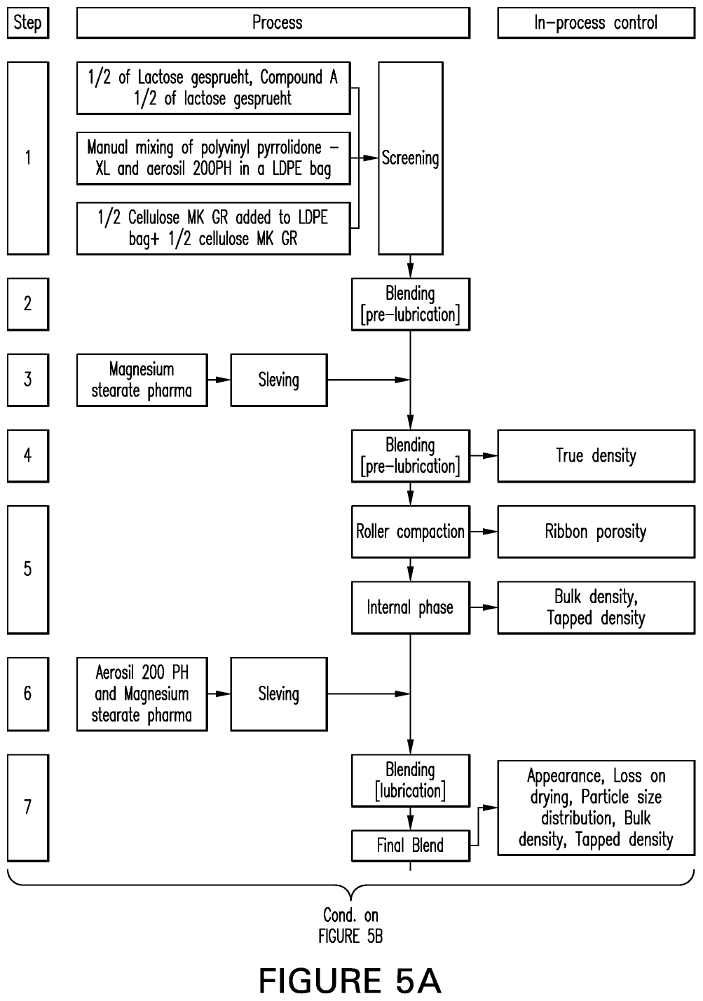

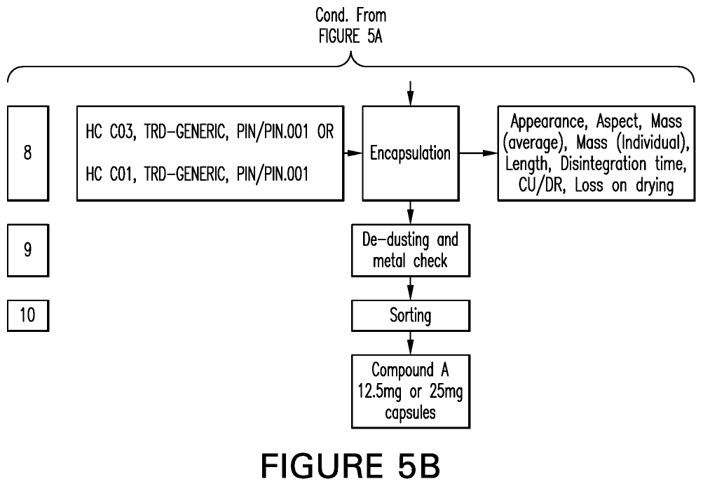

The 12.5 mg and 50 mg capsule final blends were prepared following a procedure as described in the flowchart of FIG. 5.
1. Screening to be carried out as per the following sub-steps and materials to be collected in suitable blending container.
   (a) Screen the materials in the following order as listed: ½ quantity of lactose gesprueht (spray-dried), Compound A fumarate, ½ quantity lactose spray-dried and collect the materials in the blending container.
   (b) Manually mix polyvinyl pyrrolidone XL and AEROSIL® 200PH together in a LDPE bag.
   (c) Screen this mixture of step (b) and add to materials of step (a).
   (d) Rinse the LDPE bag used in step (b) with a ½ quantity of cellulose MK GR to collect remainders of polyvinyl pyrrolidone XL & AEROSIL® 200PH if any.
   (e) Screen the portion of cellulose MK GR of step (d) alongside with remaining ½ quantity and add to mixture of step (a) in blending container.
2. Blend the mixture of step 1 in a mixer for 5 minutes.
3. Sieve magnesium stearate and add to blend of step 2.
4. Blend the mixture of step 3 in a mixer for 5 minutes.
5. Compact the lubricated blend of step 4 using roller compactor to obtain inner phase granules.
6. Screen AEROSIL® 200PH and magnesium stearate one after the other and add to inner phase of step 5.
7. Blend the mixture of step 6 in a mixer for 5 minutes to obtain lubricated blend.
8. Encapsulate the final blend of step 7 in HPMC capsule of respective size using encapsulator.
9. Perform de-dusting and metal check for filled capsules of step 8.
10. Perform weight sorting on capsules obtained in step 9.

### Example 9B: Manufacturing of 100 mg Hard, Cellulose Capsules of Compound A

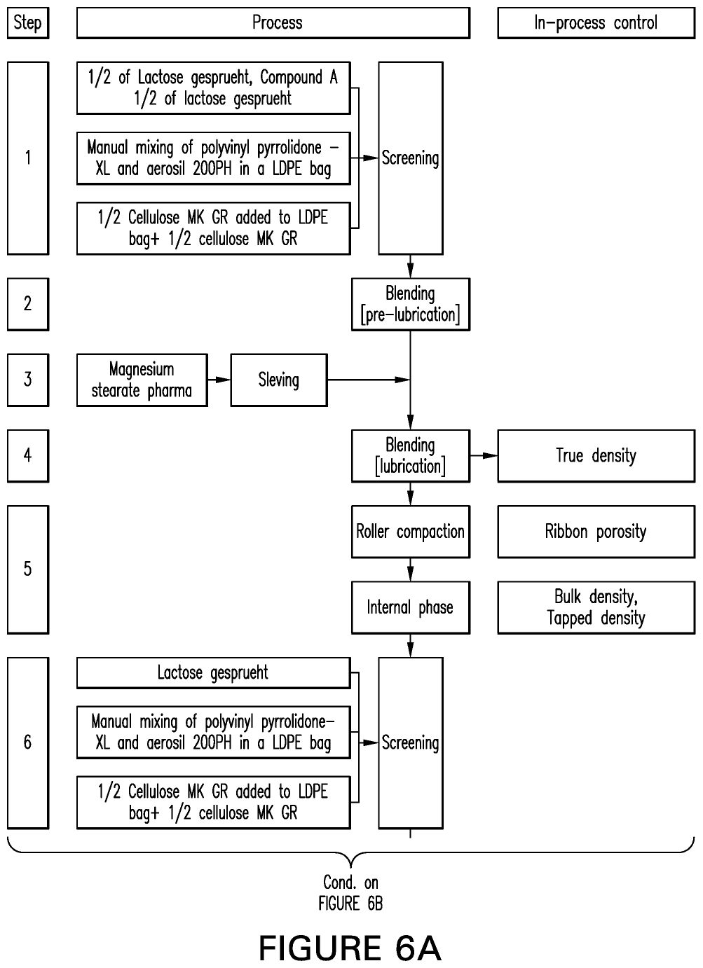

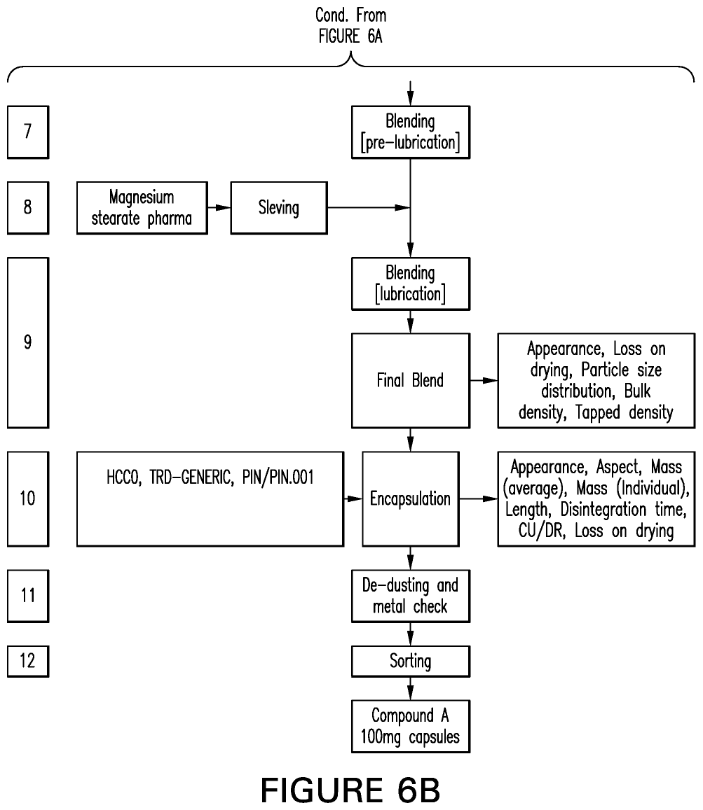

The 100 mg capsule final blends were prepared following a procedure as described in the flowchart of FIG. 6.

The 100 mg capsule final blends were prepared following a similar procedure as described in the flowchart above.
1. Screening to be carried out as per the following sub-steps and materials to be collected in suitable blending container.
   (a) Screen the materials in the following order as listed: ½ quantity of lactose gesprueht (spray-dried), Compound A, ½ quantity lactose spray-dried and collect the materials in the blending container.
   (b) Manually mix polyvinyl pyrrolidone XL and AEROSIL® 200PH together in a LDPE bag.
   (c) Screen this mixture of step (b) and add to materials of step (a).
   (d) Rinse the LDPE bag used in step (b) with a ½ quantity of cellulose MK GR to collect remainders of polyvinyl pyrrolidone XL & AEROSIL® PH200 if any.
   (e) Screen thee portion of cellulose MK GR of step (d) alongside with remaining ½ quantity and add to mixture of step (a) in blending container.
2. Blend the mixture of step 1 in a mixer for 5 minutes.
3. Sieve magnesium stearate and add to blend of step 2.
4. Blend the mixture of step 3 in a mixer for 5 minutes.
5. Compact the lubricated blend of step 4 using roller compactor to obtain inner phase granules and collect the granules in suitable container
6. Screening of outer phase materials to be carried out in following sub steps and added to the inner phase granules of step 5 to obtain mixer for pre-lubrication.
   (a) Screen lactose spray-dried and add to the inner phase granules of step 5.
   (b) Mix manually polyvinyl pyrrolidone XL and AEROSIL® 200 PH together in a LDPE bag.
   (c) Screen this mixture of step (b) and add to mixer of step 5.
   (d) Rinse the LDPE bag used in step (b) with a ½ quantity of cellulose MK GR to collect remainders of polyvinyl pyrrolidone XL & AEROSIL® 200 PH if any.
   (e) Screen thee portion of cellulose MK GR of step (d) alongside with remaining ½ quantity and add to mixture of step 5 in blending container.
7. Blend the mixture of step 6 in a mixer for 5 minutes to obtain pre-lubricated blend.
8. Sieve magnesium stearate and add to blend of step 7.
9. Blend the mixture of step 8 in a mixer for 5 minutes to obtain final blend.
10. Encapsulate the final blend of step 9 in ‘Size 0’ HPMC capsule using encapsulator.
11. Perform de-dusting and metal check for filled capsules of step 10.
12. Perform weight sorting on capsules obtained in step 11.

1. A capsule for oral administration comprising
(a) (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof,
(b) one or more fillers, and
(c) one or more disintegrants.

2. The capsule according to claim 1, wherein the drug substance is present as fumarate salt.

3. The capsule according to claim 1, wherein the drug substance is present as free base form.

4. The capsule according to claim 1, wherein one of the one or more fillers is a cellulose derivative or lactose.

5. (canceled)

6. The capsule according to claim 1, wherein one of the one ore more disintegrants is a cross-linked polyvinylpyrrolidone (PVP XL).

7. The capsule according to claim 1, comprising 3-62%, by weight of the drug substance in its free base form based on the total weight of the content of the capsule.

8. The capsule according to claim 1, comprising 20-80% by weight of the filler(s) based on the total weight of the content of the capsule.

9. The capsule according to claim 1, comprising 1-8% by weight of the disintegrant(s) based on the total weight of the content of the capsule.

10. A pharmaceutical blend comprising
(a) the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof,
(b) one or more fillers, and
(c) one or more disintegrants,
wherein the blend is manufactured by a dry process.

11. The pharmaceutical blend of claim 10, wherein the blend is manufactured by direct blending or a roller compaction process.

12. The pharmaceutical blend of claim 10, wherein said drug substance is present as fumarate salt in a polymorphic form characterized by an XRPD peak (2 theta) at 24.9±0.2°, 6.2±0.2° and 20.9±0.2°.

13. The pharmaceutical blend of claim 10, wherein said drug substance is present as free base form in a polymorphic form characterized by an XRPD peak (2 theta) at 9.7±0.2°, 18.4±0.2° and 19.4±0.2°.

14. The pharmaceutical blend according to claim 10, wherein one said filler is a cellulose derivative or lactose.

15. The pharmaceutical blend according to claim 10, wherein one said filler is lactose.

16. The pharmaceutical blend according to claim 10, wherein one said disintegrant is a cross-linked polyvinylpyrrolidone (PVP XL).

17. The pharmaceutical blend according to claim 10, comprising 3-62%, by weight of the drug substance in its free base form based on the total weight of the content of the capsule.

18. The pharmaceutical blend according to claim 10, comprising 20-80% by weight of the filler(s) based on the total weight of the content of the capsule.

19. The pharmaceutical blend according to claim 10, comprising 1-8% by weight of the disintegrant(s) based on the total weight of the content of the capsule.

20. A dry process for making the capsules according to claim 1, the process comprising a roller compaction process step.

21. The dry process for making the capsules according to claim 1 characterized by the following process steps
(a) roller compaction of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof, with one or more fillers, and one or more disintegrants, and optionally one or more additional pharmaceutical excipients, to obtain granules,
(b) blending the granules of step (a) with additional pharmaceutical excipients to obtain a pharmaceutical blend, and
(c) machine-encapsulation of the pharmaceutical blend of step (b) into capsules, preferably hard non-gelatin HPMC capsules.

22. A capsule obtainable by the dry process according to claim 20.

23-27. (canceled)

28. The capsule for oral administration according to claim 1 comprising:
(a) 3-62% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], present as fumarate salt
(b) 20-85% by weight of lactose and cellulose, and
(c) 1-8% by weight crosslinked polyvinylpyrrolidone.
based on the total weight of the content of the capsule.

29. The capsule for oral administration according to claim 1 comprising:
(a) 3-62% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], present as fumarate salt
(b) 20-85% by weight of lactose and cellulose,
(c) 1-8% by weight crosslinked polyvinylpyrrolidone,
(d) 0.1-2% by weight magnesium stearate, and
(e) 0.1-1% by weight colloidal silicon dioxide,
based on the total weight of the content of the capsule.

30. The capsule for oral administration according to claim 1 comprising an inner phase and an external phase, the inner phase comprising:
(a) 3-62% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof,
(b) 20-85% by weight of lactose and cellulose,
(c) 1-8% by weight crosslinked polyvinylpyrrolidone,
(d) 0.1-2% by weight magnesium stearate,
(e) 0.1-1% by weight colloidal silicon dioxide, and
the external phase comprising:
(f) 0.1-2% by weight magnesium stearate,
(g) 0.1-1% by weight colloidal silicon dioxide, and
based on the total weight of the content of the capsule.

31. The capsule for oral administration according to claim 1 comprising an inner phase and an external phase, the inner phase comprising:
(a) 3-62% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], or a pharmaceutically acceptable salt thereof, or a free form thereof,
(b) 20-85% by weight of lactose and cellulose,
(c) 1-8% by weight crosslinked polyvinylpyrrolidone,
(d) 0.1-1% by weight magnesium stearate,
(e) 0.1-1% by weight colloidal silicon dioxide, and
the external phase comprising:
(f) 0.1-1% by weight magnesium stearate,
(g) 0.1-1% by weight colloidal silicon dioxide, and Optionally,
(c) 1-2% by weight crosslinked polyvinylpyrrolidone,
(d) 1-10% by weight of lactose and cellulose,
based on the total weight of the content of the capsule.

32. The capsule for oral administration according to claim 1 comprising, consisting essentially of or consisting of:
(a) 10-20% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], present as fumarate salt,
(b) 70-85% by weight of lactose and cellulose,
(c) 3-8% by weight crosslinked polyvinylpyrrolidone,
(d) 0.5-1.5% by weight magnesium stearate, and
(e) 0.25-1% by weight colloidal silicon dioxide,
based on the total weight of the content of the capsule.

33. The capsule for oral administration according to claim 1 comprising, consisting essentially of or consisting of:
(a) 30-62% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], present as fumarate salt,
(b) 20-40% by weight of lactose and cellulose,
(c) 5-8% by weight crosslinked polyvinylpyrrolidone,
(d) 0.5-2% by weight magnesium stearate, and
(e) 0.5-1% by weight colloidal silicon dioxide,
based on the total weight of the content of the capsule.

34. The capsule for oral administration according to claim 1 comprising, consisting essentially of or consisting of:
(a) 50-56% by weight of the drug substance (S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline], present as fumarate salt,
(b) 20-40% by weight of lactose and cellulose,
(c) 5-8% by weight crosslinked polyvinylpyrrolidone,
(d) 0.5-2% by weight magnesium stearate, and
(e) 0.5-1% by weight colloidal silicon dioxide,
based on the total weight of the content of the capsule.

35. The capsule of claim 2, wherein the fumarate salt is in a polymorphic form characterized by an XRPD peak (2 theta) at 24.9±0.2°, 6.2±0.2° and 20.9±0.2°.

36. The capsule of claim 3, wherein the free base form is in a polymorphic form characterized by an XRPD peak (2 theta) at 9.7±0.2°, 18.4 0.2° and 19.4±0.2°.
